(19) United States
(12) Patent Application Publication
(10) Pub. No.: US 2007/0062460 A1
(43) Pub. Date: Mar. 22, 2007

(54) Swine saliva sampling method

(76) Inventor: Robert Simer, Perryton, TX (US)

Correspondence Address: WESLEY B. AMES, 7031 LOS VIENTOS SERENOS, ESCONDIDO, CA, 92029, US

(21) Appl. No.: 11/233,749

(22) Filed: Sep. 21, 2005

(51) Int. Cl. A01K 29/00

(52) U.S. Cl. 119/709

(57) ABSTRACT

A method and device for obtaining livestock saliva samples is described. An absorbent material is mounted in an animal pen such that the animal voluntarily mouths the material, thereby depositing saliva in the material. The saliva can then be extracted from the absorbent material and tested as desired.

### RELATED APPLICATIONS

NOT APPLICABLE.

### FIELD OF THE INVENTION

The present invention relates to method for obtaining saliva samples from swine and other animals.

### BACKGROUND OF THE INVENTION

The following discussion is provided solely to assist the understanding of the reader, and does not constitute an admission that any of the information discussed constitutes prior art to the present invention.

In large scale livestock operations, and in particular, swine raising operations, it is advantageous to monitor the condition of the animals for infective agents, levels and types of antibodies, and other biological parameters. Typically such monitoring is performed by immobilizing individual animals and taking samples, usually blood samples.

Such sampling has the limitations of high labor costs, long sampling time, and the risks associated with close handling of large animals. As a result, often the number of animals sampled from a large population of animals is less than the number desirable to provide appropriate statistical confidence in the testing results. This is particularly true in situations in which repeated testing over a period of time is desirable.

### SUMMARY OF THE INVENTION

The present invention concerns methods and materials for conveniently obtaining saliva samples from mammals, especially mammalian livestock and in particular swine, for conducting a variety of assays. Conventionally, assay samples are obtained from live mammals such as swine and other livestock and consist of blood, urine, and/or saliva. Typically, the taking of samples involves isolating (and often immobilizing) a single animal and obtaining the desired sample. For saliva samples, this generally involves taking a swab from the inside of the mouth.

This invention provides a more convenient method for taking such saliva samples, without the need for immobilizing individual animals, and is applicable to obtaining either individual samples or pooled samples from groups of animals such as those in common pens or pastures. As a result, the present method allows assays to be conducted for individual animals, such as to test for particular diseases or conditions, or to screen groups of animals to monitor their condition, such as monitoring for the presence of a disease in at least some animals in the group.

Thus, in a first aspect, the invention concerns a method for obtaining mammalian saliva samples such as mammalian livestock saliva samples (and in particular swine saliva samples), which involves providing a saliva sampling device that includes an absorbent material that is selected and/or configured to withstand mouthing and chewing by the animals, while absorbing saliva during such mouthing or chewing, and is located such that the animals (e.g., penned animals) to be tested can access the material. The sampling device is mounted in or adjacent to a non-immobilizing animal pen or enclosure but accessible to the animal or animals in the pen or enclosure such that the animal(s) can voluntarily mouth or chew on the absorbent material or a covering over the absorbent material. (Any mouthing or chewing either directly on the absorbent material or on an associated material (e.g., a covering) such that saliva is transferred to the absorbent material is referred to as chewing or mouthing on the absorbent material.) After the test animal(s) have mouthed and/or chewed the absorbent material, saliva is extracted from the absorbent material for assaying and/or preservation.

In particular embodiments, the absorbent material is a woven or twisted rope or strap. The material can be constructed of a natural fiber or fibers (e.g., cotton, hemp, and the like), but can also be constructed of synthetic fibers or mixed synthetic and natural fibers. Within the material, additional absorbent materials can be included, e.g., an absorbent core within a rope. Additionally, non-fibrous absorbent materials can be used. Also in certain embodiments, the sampling device is constructed with an absorbent material on or over a relatively non-absorbent material, e.g., wood or rubber material; the device includes a covering over an absorbent material, e.g., a relatively non-absorbent but water-permeable material covering an absorbent material (e.g., a porous membrane or fabric over a material such as a tampon, tampon-like material, random fiber fill, or the like).

In particular embodiments, the absorbent material is 0.5-1, 1-2, 2-3, or 3-4 feet in length; the absorbent material is in the form of a rope; the absorbent material is in the form of a strap or webbing; the absorbent material is in the form of a rope that has a diameter of 0.2-0.3, 0.3-0.4, 0.4-0.5, 0.5-0.6. 0.6-0.7, or 0.7-0.8, 0.8-1.0, 1.0-1.2, 1.2-1.5 inch or additional range defined by taking any two of the endpoints for the specified ranges as the endpoints of the additional range; the absorbent material is in the form of a strap that has a width of 0.25-0.5, 0.375-0.625, 0.5-0.75, 0.75-1.0, 1.0-1.5, 1.5-2.0 inch or an additional range defined by taking any two of the range endpoints just specified as the endpoints of the additional range, along with a thickness less than the width, e.g., a thickness of 0.125-0.25, 0.25-0.35, 0.35-0.45, 0.45-0.55, 0.55-0.75 inch or additional range defined by taking any two of the range endpoints just specified as the endpoints of the additional range. In other embodiments, the absorbent material is in the form of a core (e.g., cylindrical form such as a tampon), which is covered with a covering (e.g., a hollow cylinder), which covering is absorbent and/or water permeable, e.g., cloth, rubber, resilient plastic, woven, perforated, random direction fiber. In particular embodiments, the outer dimensions of the covering are as specified for rope or webbing above.

The sampling device can also include additional components. Examples of such additional components include attachment components, colorants, dyes (e.g., marking dyes), flavoring components, odorants, texturing (e.g., to change the mouth feel of the absorbent material), and protective covering (e.g., to protect the absorbent material from degradation from animal mouthing or chewing).

Also in certain embodiments, the sampling device or absorbent material for use in a sampling device is packaged sterilely prior to use; the sterile packaging is a sealed plastic pouch or bag; the sterile packaging is a paper pouch or bag; the absorbent material is sterilized by autoclaving, uv irradiation, and/or gas sterilization; the sample is collected in a tube or vial; a sample collection tube or vial is sterile prior to use; a sample collection tube or vial contains at least one preservative; a dry preservative is provided in a sample collection tube or vial.

In certain embodiments, the sampling device is used to obtain a sample from a single animal; a plurality of animals; at least 5, 10, 15, 20, 25, or 30 animals; the device is left in place for at least 5, 10, 15, 20, 30, 40, 50, 60, 5-10, 10-20, 20-30, 30-40, 40-60, 10-40, 5-60, 10-60, 20-60, 30-60 minutes; at least 5%, 10%, 20%, 30%, or 40% of the animals in a group of at least 100, 200, 300, 400, 500, 1000, or 2000 animals are sampled; the plurality of animals are in a common pen; the plurality of animals are in a common pasture; the plurality of animals are in a common free-range area.

In particular embodiments, a saliva sample is extracted by physical extraction, e.g., by squeezing, rolling, centrifugation, or by solvent extraction, or by a combination of solvent extraction and any physical method, such as squeezing, rolling, or centrifugation; the saliva sample is extracted from the absorbent material before drying; the saliva sample is dried and is extracted after being resuspended.

While the present methods and corresponding materials are particularly applicable to obtaining saliva samples from closely penned animals (e.g., indoor pens), it is not limited to such pens, but can also be used in large pens and enclosures (e.g., pastures), and even for free range animals. In such cases, the saliva sampling device should be mounted in a location such that it will be encountered by the animals to be tested, and should be left for sufficient time to allow contact by the desired number of animals.

In particular embodiments, the animal(s) are swine, cattle, horses, goats, sheep, dogs, cats.

In a related aspect, the invention concerns a method for assaying a livestock saliva sample. The method involves obtaining at least one saliva sample using a saliva sampling device as described herein, and assaying the sample for at least one property of the sample. In general such property is indicative of a characteristic of the animal(s) from which the sample was obtained.

In particular embodiments, the saliva sample is obtained as described in the preceding aspect and/or the sampling device is as described herein.

In a further related aspect, the invention provides a method for monitoring an animal or group of animals (e.g., mammals or livestock such as swine, cattle, horses, goats, or sheep) for a disease or condition, by obtaining an testing a saliva sample as described herein using the present saliva sampling device, and assaying the saliva sample so obtained for the a property characteristic of the presence of the disease or condition. Such disease or condition may, for example, be a viral, bacterial, or fungal infection. In particular embodiments, the method involves obtaining repeat saliva samples, e.g., repeating the sampling at least 2, 3, 5, 10, or even more times. Such repeat sampling may be repeats giving multiple samples either concurrent or within a short period of time, e.g., within 4 hours, 8 hours, 12 hours, 24 hours and/or may be obtained over an extended period of time, e.g., over a period of 2-4 days, 2-7 days, 7-14 days, 14-21 days, 21-28 days, 28-60 days, 60-180 days, or even longer, or over an additional range defined by taking any two of the range endpoints just listed as the endpoints of the new range.

In particular embodiments, the saliva sample is obtained as described in a method for obtaining saliva samples and/or using a sampling device as described herein.

In certain embodiments, the assay determines the presence of nucleic acid corresponding to an infective agent; the assay determines the presence of antibodies binding to a particular antigen; the assay determines the level of a particular type of immunoglobulin.

In another related aspect, the invention provides a livestock (e.g., swine) saliva sampling device. The sampling device includes an absorbent material, and one or more of an odorant, a flavoring agent, and a marking dye, e.g., an odorant and a flavoring agent (which may be the same or different), an odorant and a marking dye, a flavoring agent and a marking dye.

In yet another aspect, the invention provides a kit for obtaining and/or obtaining testing and/or testing a livestock saliva sample. The kit includes at least one saliva sampling device and instructions for using the sampling device to obtain a livestock saliva sample. Alternatively, the kit includes at least one saliva sampling device and a saliva extractor. In yet another alternative, the kit includes at least one saliva sampling device and a container for saliva sample collection. The saliva sampling device is a sampling device as described herein for the present invention.

In yet another aspect, the invention concerns instructions in written (e.g., in character and/or pictorial form on a solid medium such as paper, cardboard, plastic, or metal) or electronic form (e.g., in computer memory, in a computer accessible data storage medium, and/or displayed on a computer display device.

The term “absorbent material” means a material that, due to its composition and construction will absorb and retain at least 15% water weight/weight or at least 20% water weight/volume following immersion for 3 minutes at room temperature, and will retain such percentage water for at least 5 minutes despite draining under gravity. Absorbent material may absorb and retain more, e.g., at least 20, 30, 40, 50, 60, 70, 80, 90 percent or even more (w/w or w/v).

In connection with the present livestock saliva sampling devices, the phrase “adapted for mounting” indicates that the device includes a component or components that are specifically selected or configured to be suitable for mounting for access by the animal(s) to be sampled, where the mounting method is not simply tying of the absorbent material itself (e.g., tying the rope itself).

As used herein, the term “assaying” refers to a process or technique for determination of a biochemical parameter of a biological sample, e.g., a saliva sample.

The term “attractive to swine” means, in relation an object that on average and following initial contact, swine will exhibit significantly more interest toward the object than toward a non-attractive or neutral object.

In connection with assays to animals, and in particular livestock, the term “characteristic” refers to a biological property, state, or condition of an animal or group of animals. Examples include viral or bacterial infection, immunological status (e.g., level of an immunoglobulin(s)), level of an enzyme, and the like.

In the context of an animal contacting a saliva collection device, the term “chewing” refers to a repeated biting action, with or without release between bites.

With respect to additives to the present saliva collection devices, the term “flavoring agent” refers to a chemical compound or mixture of compounds that imparts a flavor detectable, noticeable, and attractive to an animal to be sampled. Advantageously, such an agent is an attractive flavor such that, on average, such animal will spend more time mouthing or chewing a device with the flavoring agent than an identical device without the flavoring agent.

The term “livestock” is used to refer to domesticated animals typically raised for agricultural purposes, such as cattle, horses, sheep, goats, and swine.

In relation to confinement pens for livestock, the term “non-immobilizing” means that the animal is not restricted to one position, e.g., is not in a squeeze chute or the like, and is free to walk about within the pen.

As used herein in connection with contact between an animal and one of the present devices, the term “mouthing” refers to the animal taking a portion of the device into its mouth without chewing.

As used in connection with the present invention the term “odorant” refers to a material that has an odor that is detectable, noticeable, and attractive to the animal from which a saliva sample is to be obtained. Advantageously, the odorant is attractive to the animal, such that the animal will interact with an object having the odorant sooner, more often, and/or for a longer period than the same object without the odorant.

In connection assaying of biological samples, the term “property” is used to refer to a determinable biochemical parameter of the sample material. Examples include presence of infective agent, level of a nucleic acid, level of an immunoglobulin, level of an enzyme or other protein, and the like.

The term “rope” is used to refer to a heavy line, that is, an elongated fibrous material, generally twisted, woven, or braided. Common materials include cotton, hemp, nylon, sisal, and the like.

As used herein, the term “saliva” refers to oral fluids of a mammal. Unless expressly indicated, the term “saliva” is not limited to secretions from salivary glands.

As used in connection with the present invention, the terms “saliva extractor” and “saliva extraction device” means a device adapted to remove saliva from an absorbent material, e.g., a press, wringer, clamp, centrifuge.

In connection with biological materials, the term “sample” refers to a relatively small amount of a biological material, suitable for biochemical testing.

As used herein the term “swine” is used synonymously with “hogs” and “pigs” to refer to porcine animals.

In connection with an item constructed of an absorbent material applicable to the present invention, the terms “strapping” and “webbing” refers to a configuration of absorbent material (usually fibrous material, e.g., woven or braided) in which the item is elongated and the width of the item in cross-section is significantly greater than the thickness.

In connection with contact between an animal and a present sampling device, the term “voluntarily” means that the animal contacts the device of its own volition. This is distinguished, for example, from a tester taking a sample by inserting an absorbent material such as a swab into the mouth of an animal to be tested.

Additional embodiments will be apparent from the Detailed Description and from the claims.

### DETAILED DESCRIPTION OF THE PREFERRED EMBODIMENTS

The present swine saliva sampling methods and devices provide enhanced convenience and safety for obtaining such saliva samples. For simplicity in view of the various embodiments of the present invention, the present system and method for obtaining livestock saliva samples can by referred to as the ROPEM SYSTEMTM™.

Construction of Sampling Devices

The present sampling devices and associated components (i.e., the ROPEM SYSTEM™) can be constructed in a variety of ways, and using different materials. In general, the devices are constructed such that they can be mounted in, on, or adjacent to a hog pen, where a hog or hogs in the pen can access and mouth or chew on the device. The device is constructed of or includes an absorbent material that absorbs and contains saliva from the hog(s) when the animal mouths or chews on it.

The type of absorbent material and its configuration can be varied so long as the material will withstand mouthing or chewing by the hogs sufficiently to allow the device to absorb saliva and then to be recovered and saliva extracted. For example, the absorbent may be in the form of a woven or twisted rope, a woven strap, or a random fiber orientation such as a felted material.

The sampling device can be as simple as a section of absorbent rope tied to a pen barrier, but may also have additional advantageous features.

For example, the device can include a mounting device, such as a clip clamp, tie, or bracket, e.g., constructed of plastic, metal, or combination thereof.

Even though simple rope or straps can be used, other configurations can be advantageous. For example, an absorbent material can be placed inside a water permeable cover, or an absorbent material may be placed over a solid core, e.g., a solid rod-shaped core. Additional configurations can also be constructed and used.

In addition, the device can be modified to be more attractive to the animals, more effective at absorbing and/or retaining saliva, more durable, to mark animals that contact the device, or to have other advantageous property.

For example, a saliva sampling device may have a flavoring agent added, such that the animals to be tested will find the device more attractive. The result is that the animals will access the device sooner, more frequently, for longer periods, and/or with more vigor. This can be advantageous, for example it can result in greater saliva sample volumes and/or saliva from an increased number of animals. Exemplary flavoring agents include attractive food items or flavors of such food items, or sweetening agents such as sugar or high sugar content materials. An example of such an agent useful for swine is simple JELLO®. Those skilled in the art are familiar with or can readily select attractive flavoring agents for the different types of animals.

Likewise, odorants may be added. Such odorants may be part of a flavoring agent, or may be distinct agents. Similar to flavoring agents, such odorants are attractants for the animals to be sampled. Thus, for example, an animal in close proximity may be induced to interact with the saliva sampling device more quickly, for greater periods of time, with greater frequency, or with greater vigor. As with flavoring agents, those skilled in the art are familiar with or can readily select attractive odorants for the different types of animals.

The devices may also include a marking dye, such that animals chewing on the device are temporality marked with the dye, thereby identifying animals that have chewed on the device and deposited saliva. Such dye can be selected to be staining and persistent but non-permanent.

The device may also be constructed such that a texturing material is placed on the outside so that the animals encounter a texture that the animal finds interesting so that the animals will voluntarily chew on the device or will chew for longer periods of time.

Use of Sampling Devices

Consistent with the basic construction of the sampling devices, the use of the devices involve appropriate placement of the device in or near an animal enclosure or pen, e.g. a hog pen where the animal(s) (e.g., hog or hogs) in the pen can readily reach the device to mouth or chew on it. Thus, the device is typically placed where the animals in the pen will readily notice and be attracted to the device. For example, the device can be suspended on the inside of a pen fence or barrier, e.g., at about shoulder height of the animals being sampled.

Alternatively, a saliva sampling device may be mounted on or to a surface within the pen where it will be readily encountered by the animal(s), suspended over a portion of the pen, in or adjacent to a feed trough, or other such locations accessible to the animals.

Extraction and Use of Saliva Samples

A number of different methods for extracting the saliva sample can be utilized, e.g., depending on the type and size of the absorbent material in the sampling device. At least two basic approaches can be used, those being mechanical extraction, and solvent extraction.

In mechanical extraction, the absorbent material is squeezed and/or centrifuged, thereby removing a portion of the liquid saliva retained in the material. For example, the material may be squeezed by twisting and/or pressing the material, e.g., using a roller (pressed against a hard surface) or pair of rollers (e.g., a wringer type assembly), clamping between two hard surfaces, or squeezing and pulling the material by the closure of a sample tube or vial.

In solvent extraction, the absorbent material or portion thereof is soaked and/or washed with a solvent, e.g., water or saline solution. The solvent so used then contains the saliva. Such solvent extraction can be combined with mechanical extraction, e.g., to improve recovery of the saliva captured in the absorbent material.

A container used to collect the saliva sample can be sterile. In addition, or alternatively, the container may include a preservative to prevent or limit degradation of materials in the sample. Such degradation may, for example, occur due to growth of bacterial or fungal contaminants, or to the presence of proteases or nucleases. Alternatively, such preservative may be added separately, or may be included in a solvent used to extract the saliva sample. Any of a large variety of containers can be used, but are typically vials, tubes, or bottles with secure closures, and are usually made of plastic or glass.

Even though in most cases the saliva sample will be extracted within minutes or a few hours after the sampling period, this is not essential. In some cases it will be desirable to retain the sampling device with the saliva sample for a longer period of time. For example, the sampling device (or portion thereof) with saliva sample in the absorbent material can be retained for later testing, and/or can be sent to a remote testing laboratory or other testing entity. Such laboratory or other entity can then extract the sample (from either a wet or dry sampling device) and carry other the assay(s).

Alternatively, in some cases the assay can be carried out directly on the sampling device without sample extraction.

Kits

The present sampling devices can be provided in kit form adapted for carrying out or assisting in carrying out the present methods. Such kits can be configured in various ways. Without limitation, kit components can include one or more saliva sampling devices (which may be sterile or sterile packaged devices), instructions for mounting a saliva sampling device and/or for obtaining saliva samples using a saliva sampling device, a saliva extraction device, a saliva collection container, sample preservative, wash solution, marking dye, water impermeable gloves (e.g., sterile gloves, such as PVC, silicon rubber, or rubber gloves), mailing containers and/or mailing labels (e.g., addressed to a testing laboratory) and/or assay components. Such kits may be packaged such that all components of the kit are in a single container, e.g., in a common box, bag or pouch. Such kits may contain components for carrying out a single sample collection (i.e., a single use kit) or may contain components to carry out a plurality of such sample collections (a multi-use kit), e.g., 2, 3, 4, 5, 6, 7, 8, 9, 10, 2-4, 4-6, 6-8, 8-10, 10-12, 2-10, 10-20, 20-100, at least 4, at least 10, at least 20, at least 40, at least 80, or at least 100 such sample collections.

Thus, in some cases, a kit includes at least a saliva sampling device and instructions for use of such device to obtain a saliva sample(s). Such kit can also include additional components, such as one or more of those described above. For example, the kit can also include a saliva extraction device and/or a saliva sample collection container and/or a preservative and/or a wash solution and/or marking dye and/or assay components. In addition, or alternatively a kit can include one or more other non-listed components.

In other cases, a kit includes at least a saliva sampling device and a saliva extraction device. Advantageously the kit also can include instructions for use of the kit or for obtaining saliva samples. As above, the kit can also include one or more of the other listed components, and/or other components not listed. In certain embodiments, the kit includes each combination of the listed components taken, 2, 3, 4, 5, 6, 7, 8, or 9 at a time.

In yet other cases, a kit includes at least a saliva sampling device and a sample collection container. Advantageously the kit can also include instructions for use of the kit or for obtaining saliva samples. Again as above, the kit can also include one or more of the additional listed components, and/or other components not listed.

In most cases, instructions for use will be in printed form, e.g., as characters and/or pictorial images on paper, plastic, or metal substrate. In addition, instructions can be provided in electronic form, e.g., in computer memory (e.g., random access memory), recorded on a computer hard drive, floppy drive, CD, DVD, flash memory device, or other recordable data storage device. The instructions may also be displayed on a computer display device, e.g., a computer monitor. The instructions may form part of a web site, or be linked to a web site.

### EXAMPLES

The following examples illustrate initial evaluation and use of suspended absorbent material as saliva collection devices in the ROPEM SYSTEM™.

### Example 1

### Material Selection for Saliva Sample Collection

Various ropes were evaluated as absorbent materials suitable for use as livestock saliva collection devices. These included 5 different materials; sisal, cotton (2 diameters), manila, nylon, and sash cord. These materials were evaluated for their abilities to absorb water as an indicator of their abilities to absorb and hold saliva from livestock, in particular from swine.

A one foot section of each material was initially weighed, submerged in water for 3 minutes, and weighed again following the submersion. The difference in weight reflected the amount of retained water. Normalized to 1 ounce of dry rope, the materials retained the following amounts of water:

| Material | Oz retained water/oz rope |
|---|---|
| Sisal ½ inch | 0.90 |
| Cotton ⅝ inch | 0.63 |
| Cotton ¾ inch | 0.59 |
| Sash Cord ¼ inch | 0.56 |
| Nylon ⅝ inch | 0.47 |
| Manila ⅝ inch | 0.28 |

The tested materials all exhibited substantial water retention, and could be used for saliva collection. The table above demonstrates that the tested sisal and cotton materials exhibited better water retention than the nylon and manila materials. Thus, these materials can advantageous due to their higher water retention capacity.

### Example 2

### Determination of Swine Chewing on Suspended Cotton Rope

Based results from the water retention determinations and preliminary testing for swine contact with articles presented in pens in swine nursery barns with 6-9 week old pigs, cotton rope was used to determine the time for a statistically acceptable percentage of swine in a pen to chew on the rope.

Six to nine week old pigs in weekly cohorts were held in pens in commercial nursery barns with 25 pigs per pen. Three foot sections of cotton rope were suspended from pen fences. Individual animal interactions with the rope were observed by a person from a distance of 10 feet, and the interacting animals were marked with colored wound spray following an interaction threshold time. Interaction was set as 5 chews or 5 seconds of mouthing, whichever occurred first.

It was found that the time for at least 10 pigs to interact with the suspended rope ranged from 10 to 28 minutes, with a mean of 18.33 minutes. Thus, at least 40% of the animals in each pen interacted with the rope within 30 minutes.

Interaction time was also determined for rope placed on pen floors. It was found that the average time for 10 pigs to interact with the rope was less than for the suspended rope, with a mean of 15.50 minutes, with a range of 10-17 minutes. While the floor placement yielded effective animal interaction, it is less advantageous than the suspended rope due to fecal contamination and the potential displacement of the rope out of the pen or into other inaccessible location. Nonetheless, these results demonstrate that a variety of placement locations can be effective for animal interaction, e.g., suspension over the pen, mounting on an object within the pen, mounting along a fence, as well as others.

### Example 3

### Determination of Porcine Reproductive and Respiratory Syndrome Virus (PRRSV) Presence by PCR from Rope-Collected Swine Saliva Sample

Saliva collection using cotton rope suspended from pen fences was used in a PCR assay to determine the presence of PRRSV. PPRSV was found by PCR assay in four out of four saliva samples tested.

Pigs were housed on coated plastic flooring in four temperature controlled, power ventilated, commercial nursery barns, with 2 rooms per building. One foot of ¾ inch twisted cotton rope was attached to the fencing confining 50 day old swine weighing approximately 45 pounds and had been in the nursery for 6-7 weeks. The ropes were attached in the middle of the front aisle gate of four nursery pig rooms using plastic zip ties at approximately shoulder height for the pigs. The ropes were left in place for 90 minutes and animals were allowed to freely interact with the rope.

Following the 90 minute interaction period, the ropes were removed from the pens, placed in individual sealable plastic bags and transported to an office. Each rope was squeezed to extract separate saliva samples, yielding 1-6 mL per rope. The extracted saliva samples were placed in separate snap top plastic serum tubes, and send for analysis. All four saliva samples were found positive for PRRSV using a PCR assay.

As a control, one pig from each room was euthanized and tested for PRRSV antibody by ELISA, and for the presence of PRRSV in serum and tissue samples. The euthanized pigs were found to be negative for PRRSV antibody in the ELISA tests, but were positive for PRRSV in the serum and tissue samples.

The tests confirm that saliva samples obtained using the ROPEM SYSTEM™ can effectively be used to test for the presence of particular viruses, even in cases where ELISA antibody tests may be negative. In addition, the pooled nature of the sample enhances the screening capability and provides enhanced statistical confidence.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to the materials used and shape of the absorbent material. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

Thus, additional embodiments are within the scope of the invention and within the following claims.

What is claimed is:

1. A method for obtaining a swine saliva sample, comprising
providing to at least one swine in a non-immobilizing pen a saliva sampling device comprising an absorbent material able to withstand swine mouthing, wherein said saliva sampling device is mounted such that the sampling device is accessible to said swine from within said pen; and
collecting saliva from said sampling device following mouthing by said swine.

2. The method of claim 1, wherein said absorbent material is rope.

3. The method of claim 1, wherein said absorbent material is strapping.

4. The method of claim 1, wherein said sampling device further comprises at least one flavoring agent.

5. The method of claim 1, wherein said sampling device further comprises at least one odorant.

6. The method of claim 1, wherein said sampling device further comprises a marking dye.

7. The method of claim 1, wherein said sampling device is mounted in said pen for 5-60 minutes.

8. The method of claim 1, wherein said sampling device is mouthed or chewed by at least 10 swine.

9. The method of claim 1, wherein said saliva is collected from said absorbent material by mechanical extraction.

10. The method of claim 1, wherein said saliva is collected from said absorbent material by solvent extraction.

11. The method of claim 1, wherein said sampling device is provided to a single swine.

12. The method of claim 1, wherein said sampling device is provided to at least 20 animals in a common enclosure.

13. A swine saliva sampling device, comprising
an absorbent material configured to withstand swine mouthing or chewing, having attached thereto a mount for attaching in or adjacent to a hog pen; and
at least one flavoring agent or odorant attractive to swine on said absorbent material.

14. A swine saliva sampling kit, comprising
a swine saliva sampling device comprising an absorbent material configured to withstand swine mouthing or chewing; and
instructions for use for obtaining a swine saliva sample.

15. The kit of claim 14, further comprising a sample collection container.

16. The kit of claim 14, wherein said sampling device is sterile.

17. The kit of claim 14, further comprising a saliva extractor for squeezing said absorbent material.

18. The kit of claim 14, further comprising a wash solution.
